(19) United States
(12) Patent Application Publication
(10) Pub. No.: US 2026/0237060 A1
(43) Pub. Date: Aug. 13, 2026

(54) METHOD AND DEVICE FOR ASSESSING PUBERTAL ELBOW BONE AGE USING LATERAL ELBOW X-RAY IMAGES AND ARTIFICIAL INTELLIGENCE MODEL

(71) Applicant: KOREA UNIVERSITY RESEARCH AND BUSINESS FOUNDATION, Seoul (KR)

(72) Inventors: Ga Young CHOI, Ansan-si, Gyeonggi-do (KR); Sung Won HAM, Ansan-si, Gyeonggi-do (KR); Bo Kyung JE, Ansan-si, Gyeonggi-do (KR)

(21) Appl. No.: 19/152,395

(22) PCT Filed: Jan. 30, 2024
(86) PCT No.: PCT/KR2024/001416

§ 371 Date: Jul. 30, 2025

(30) Foreign Application Priority Data
Jan. 31, 2023 (KR) 10-2023-0012924
Dec. 29, 2023 (KR) 10-2023-0197113

(51) Int. Cl. G06T 7/00; G06T 7/11

(52) U.S. Cl. CPC G06T 7/0012; G06T 7/11; G06T 2207/10116; G06T 2207/20036; G06T 2207/30008

(57) ABSTRACT

The present disclosure relates to a bone age assessment method performed by a bone age assessment device, the method including (a) acquiring a lateral elbow X-ray image, (b) segmenting an olecranon region from the X-ray image, (c) analyzing morphological features of an olecranon ossification center in the segmented olecranon region, and (d) determining a bone age based on the analyzed morphological features.

### TECHNICAL FIELD

The present disclosure relates to a bone age assessment technique, and more specifically, to a bone age assessment method and device using a lateral elbow X-ray image of an adolescent child.

### BACKGROUND ART

Bone age assessment of children and adolescents is important for accurately assessing a skeletal maturity of a patient and may differ from a patient's chronological age. The most widely used method for assessing the bone age is a hand and wrist bone age assessment method, but this method has the disadvantage of being challenging during puberty due to minimal changes in carpal bones in this period and having a large gap between assessment ages, which limits its clinical application. Meanwhile, elbow shows characteristic changes in early puberty and typically complete fusion by the end of the first two years of puberty. Therefore, a method using elbow X-ray images is valuable for assessing bone age in children and adolescents during puberty.

The first two years of puberty are the acceleration phase, which shows a growth spurt, and peak height velocity (PHV)—the period of greatest height growth during puberty—and the sexual maturity corresponding to Tanner stage 2 serves as an indicator of the onset. During this period, the ossification process of an olecranon apophysis illustrates very characteristic morphological changes, and when the ossification process of the olecranon apophysis is completed, the acceleration phase, which is early puberty, ends and the deceleration phase, which is late puberty, begins. Therefore, when bone age is determined using the ossification process of the olecranon apophysis, which changes rapidly, an accurate assessment of the period of early puberty with rapid growth is possible.

The existing elbow bone age assessment methods using the ossification process of the olecranon apophysis include the method of Sauvegrain et al. and the method of Dimeglio et al. The method of Sauvegrain et al. is the most traditional and widely used, but it requires two X-ray images corresponding to the anteroposterior and lateral view of the elbow, which results in increased radiation exposure. In addition, it has the disadvantage of being complicated and time-consuming to interpret because the bone age is determined by assessing the morphology of each part of the elbow and summing the scores. The method of Dimeglio et al. requires only one X-ray image corresponding to the lateral view of the elbow, but it has the disadvantage of many limitations and inaccuracies in actual clinical application because it does not reflect the diversity of the ossification process of the olecranon apophysis.

### PRIOR ART LITERATURE

### Patent Literature

(Patent literature 1) Korean Patent Publication No. 10-2019-0142234

### DISCLOSURE

### Technical Problem

An object of the present disclosure is to provide a bone age assessment method and device that morphologically subdivides steps of ossification center formation and fusion seen in a maturation process of an olecranon apophysis that can be confirmed in a lateral elbow X-ray during puberty and reflects various and characteristic changes.

Objects of the present disclosure are not limited to the problems mentioned above, and other objects not mentioned may be clearly understood by those skilled in the art from the description below.

### Technical Solution

In order to achieve the object, a first aspect of the present disclosure is a bone age assessment method performed by a bone age assessment device, the bone age assessment method including: (a) acquiring a lateral elbow X-ray image; (b) segmenting an olecranon region from the X-ray image; (c) analyzing morphological features of an olecranon ossification center in the segmented olecranon region; and (d) determining a bone age based on the analyzed morphological features.

Preferably, the step (b) may include predicting a bone region in the X-ray image on a pixel basis, and segmenting a region corresponding to the olecranon in the X-ray image according to a result of the pixel-based prediction.

Preferably, the step (c) may include determining a region for the olecranon ossification center in the olecranon region, and determining the morphological features of the olecranon ossification center.

Preferably, the step (d) may include classifying the olecranon bone age according to a preset age-specific olecranon characteristic criteria based on the morphological features of the olecranon ossification center.

Preferably, the preset age-specific olecranon characteristic criteria may be set in 6-month units from 9.5 to 13 years old in girls, and in 6-month units from 11.5 to 15 years old in boys.

Preferably, in the preset age-specific olecranon characteristic criteria, the olecranon characteristic criteria may be further set for 11.25 years olds in girls and 13.25 years old in boys.

Preferably, the preset age-specific olecranon characteristic criteria may be classified into a first ossification process and a second ossification process according to the order of fusion of an accessory ossification center and the ossification center of the olecranon.

Preferably, the first ossification process may include a small olecranon ossification center step in which a height of an olecranon body is 20% or more and less than 50%, an enlarged olecranon ossification center step in which the height of the olecranon body is 50% or more, an appearance step of an accessory ossification center, a fusion step of the olecranon ossification center and the accessory ossification center, an enlargement step of the fused ossification center, a partial fusion step of less than 50% of an olecranon apophysis, a partial fusion step of 50% or more of the olecranon apophysis, and a complete fusion step of the olecranon apophysis.

Preferably, the bone age assessment method may further include an appearance step of another accessory ossification center after the fusion of the olecranon ossification center and the accessory ossification center between the fusion step of the olecranon ossification center and the accessory ossification center and the enlargement step of the fused ossification center.

Preferably, the second ossification process may include a small olecranon ossification center step in which the height of the olecranon body is 20% or more and less than 50%, an enlarged olecranon ossification center step in which the height of the olecranon body is 50% or more, an appearance step of an accessory ossification center, a fusion step of the accessory ossification center and the olecranon body, an enlargement step of the fused accessory ossification center-olecranon body and the unfused olecranon ossification center, a partial fusion step of less than 50% of the olecranon ossification center and the fused accessory ossification center-olecranon body, a partial fusion step of 50% or more of the olecranon apophysis, and a complete fusion step of the olecranon apophysis.

Preferably, the bone age assessment method may further include an appearance step of another accessory ossification center after the fusion of the olecranon ossification center and the accessory ossification center between the fusion step of the accessory ossification center and the olecranon body and the enlargement step of the fused accessory ossification center-olecranon body and the unfused olecranon ossification center.

In order to achieve the above object, a second aspect of the present disclosure is a bone age assessment device, including: an image acquisition unit for acquiring a lateral elbow X-ray image; a region segmentation unit for segmenting an olecranon region from the X-ray image; a morphological analysis unit for analyzing morphological features of an olecranon ossification center in the segmented olecranon region; and a bone age determination unit for determining bone age based on the analyzed morphological features.

Preferably, the region segmentation unit may predict a bone region in the X-ray image on a pixel basis and segment the region corresponding to the olecranon in the X-ray image according to a result of the pixel-based prediction.

Preferably, the morphological analysis unit may determine a region for the olecranon ossification center in the olecranon region, and determine the morphological features of the olecranon ossification center.

Preferably, the bone age determination unit may classify the olecranon bone age according to a preset age-specific olecranon characteristic criteria based on the morphological features of the olecranon ossification center.

A third aspect of the present disclosure for achieving the above object is a computer program stored in a computer-readable medium, in which a command of the computer program is executed, the bone age assessment method is performed.

### Advantageous Effects

As described above, according to the present disclosure, since one X-ray image corresponding to the lateral surface of the elbow is used, there is an effect of reducing radiation exposure during examination, and there is an effect of widening and subdividing the range of age that can be determined.

In addition, since the characteristic morphological changes of the olecranon apophysis that can be observed in the lateral elbow X-ray image are reflected and classified, it is effective in assessing the bone age more accurately in various clinical cases when determining the bone age in children and adolescents during puberty.

In addition, it can play a key role in assessing the musculoskeletal maturity of patients through accurate assessment of pubertal bone age and determining the timing of diagnosis and treatment for various endocrine diseases, short stature, fractures, scoliosis, or lower limb length discrepancy.

In addition, it has the effect of improving the work process of radiologists, enabling simpler and more intuitive judgments, and utilizing AI programs to assist the work of radiologists, pediatricians, or orthopedic surgeons and enable quick interpretation.

### DESCRIPTION OF DRAWINGS

FIG. 1 is a block diagram of a bone age assessment method according to a preferred embodiment of the present disclosure.

FIG. 2 is a flowchart illustrating the bone age assessment method according to one embodiment.

FIG. 3 is an exemplary diagram explaining the bone age assessment method according to one embodiment.

FIG. 4 is an exemplary diagram explaining segmentation of an olecranon region according to one embodiment.

FIG. 5 is an exemplary diagram explaining analysis of morphological features of an ossification center according to one embodiment.

FIG. 6 and FIG. 7 are exemplary diagrams explaining a deep learning algorithm according to one embodiment.

FIG. 8 and FIG. 9 are exemplary diagrams explaining determination of bone age according to one embodiment.

FIG. 10 is a diagram explaining performance of the bone age assessment method according to one embodiment.

### BEST MODE

Hereinafter, the advantages and features of the present disclosure, and the method for achieving them will become clear with reference to the embodiments described in detail below together with the accompanying drawings. However, the present disclosure is not limited to the embodiments disclosed below, but may be implemented in various different forms, and the present embodiments are provided only to make the disclosure of the present disclosure complete, and to fully inform a person having ordinary skill in the art to which the present disclosure belongs of the scope of the disclosure, and the present disclosure will be defined only by the scope of the claims. Like reference numerals refer to like elements throughout the specification. “And/or” includes each and every combination of one or more of the mentioned items.

Although the terms first, second, or the like are used to describe various elements, components, and/or sections, it is to be understood that these elements, components, and/or sections are not limited by these terms. These terms are merely used to distinguish one element, component, or section from other elements, components, or sections. Thus, it should be understood that a first element, a first component, or a first section referred to hereinafter may also be a second element, a second component, or a second section within the technical spirit of the present disclosure.

In addition, the identifiers (for example, a, b, c, or the like) for each step are used for convenience of explanation and do not describe the order of each step, and each step may occur in a different order than stated unless the context clearly indicates a specific order. That is, each step may occur in the same order as stated, may be performed substantially simultaneously, or may be performed in the opposite order.

The terminology used in the present disclosure is for the purpose of describing embodiments only and is not intended to be limiting of the disclosure. As used in the present disclosure, the singular includes the plural unless the context clearly dictates otherwise. The terms “comprise” and/or “comprising” as used in the present disclosure do not exclude the presence or addition of one or more other components, steps, operations and/or elements.

Unless otherwise defined, all terms (including technical and scientific terms) used in this specification may be used with a meaning that may be commonly understood by a person of ordinary skill in the art to which the present disclosure belongs. In addition, terms defined in commonly used dictionaries shall not be ideally or excessively interpreted unless explicitly specifically defined.

In addition, when describing embodiments of the present disclosure, if it is judged that a specific description of a known function or configuration may unnecessarily obscure the gist of the present disclosure, the detailed description will be omitted. In addition, the terms described below are terms defined in consideration of functions in the embodiments of the present disclosure, and these may vary depending on the intention or custom of the user or operator. Therefore, the definitions should be made based on the contents throughout this specification.

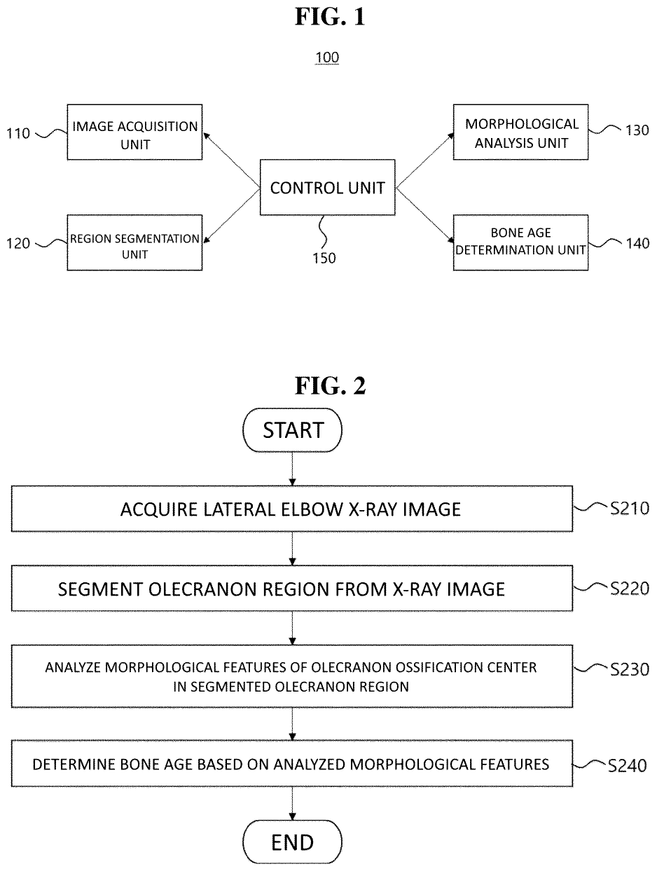

FIG. 1 is a block diagram illustrating a bone age assessment device according to a preferred embodiment of the present disclosure.

A bone age assessment device 100 is a device for performing a bone age assessment method according to the present disclosure, which receives an X-ray image of the lateral surface of the elbow and analyzes the shape of the bone to assess the bone age. That is, the bone age assessment device 100 assesses the bone age of adolescent children and adolescents by utilizing the characteristics of the characteristic olecranon ossification process during an acceleration phase corresponding to the first two years of puberty.

Preferably, the bone age assessment device 100 may be a computer that may install and execute an application or program for performing the bone age assessment method, and may be equipped with a user interface so that input and output of data may be controlled. Here, a computer means all kinds of hardware devices including at least one processor, and according to an embodiment, it may be understood to encompass a software configuration operating on the corresponding hardware device. For example, a computer may be understood to encompass all of a smartphone, a tablet PC, a desktop, a laptop, and a user client and an application running on each device, but is not limited thereto.

Referring to FIG. 1, the bone age assessment device 100 includes an image acquisition unit 110, a region segmentation unit 120, a morphological analysis unit 130, a bone age determination unit 140, and a control unit 150. Here, the control unit 150 controls the operations of the image acquisition unit 110, the region segmentation unit 120, the morphological analysis unit 130, and the bone age determination unit 140 and the flow of data.

The image acquisition unit 110 acquires an X-ray image. Preferably, the X-ray image may be captured from a separate photographing device connected to the bone age assessment device 100 by wire or wirelessly and transmitted to the bone age assessment device 100, or the X-ray image may be captured from a photographing device equipped in the bone age assessment device 100 and transmitted to the image acquisition unit 110.

The region segmentation unit 120 segments an olecranon region, which is a region necessary for determining bone age, from the X-ray image acquired from the image acquisition unit 110. Preferably, various deep learning algorithms may be applied to the method of segmenting the olecranon region.

The morphological analysis unit 130 analyzes the morphological features of the bones in the olecranon region. Preferably, the morphological analysis unit 130 may determine the region for the olecranon ossification center using a deep learning algorithm and analyze the morphological features of each bone constituting the olecranon ossification center.

The bone age determination unit 140 determines the bone age based on the morphological features of the bone. Preferably, the bone age determination unit 140 may determine the bone age based on bone age determination criteria in which the bone age is matched with the preset morphological features of the bone.

The operations performed through each configuration of the bone age assessment device 100 illustrated in FIG. 1 will be described in detail with reference to FIG. 2 below. Each step to be described with reference to FIG. 2 is described as being performed by different configurations, but is not limited thereto, and at least some of the steps may be performed in the same or different configurations depending on the embodiment.

FIG. 2 is a flowchart illustrating a bone age assessment method according to one embodiment.

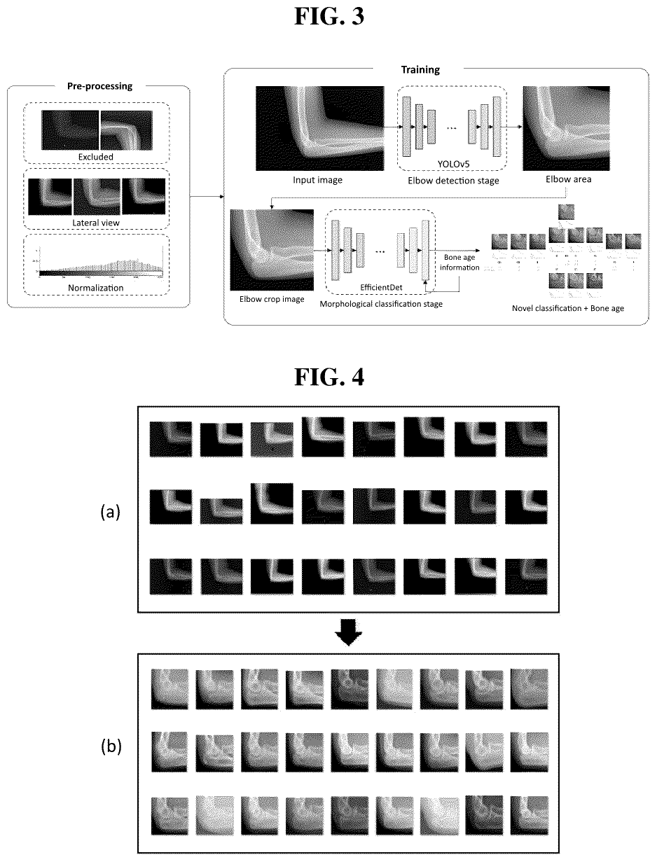

Referring to FIG. 2, the image acquisition unit 110 acquires a lateral elbow X-ray image (Step S210). Preferably, the image acquisition unit 110 may pre-process the X-ray image, referring to FIG. 3. That is, the image acquisition unit 110 may exclude an unnecessary region from the X-ray image, recognize and select a lateral view, and normalize the X-ray image.

The region segmentation unit 120 segments the olecranon region from the X-ray image (Step S220). Preferably, the region segmentation unit 120 may apply a deep learning algorithm to the X-ray image to predict the region of interst (olecranon) on a pixel basis, and segment the region corresponding to the olecranon from the X-ray image according to the result of the pixel-based prediction. More specifically, referring to FIG. 3, the region segmentation unit 120 may detect a region of interst corresponding to the olecranon by applying YOLOv5, which is a deep learning algorithm, to the X-ray image, which is an input image. The region segmentation unit 120 may automatically crop an image for the olecranon region from the X-ray image by applying a YOLOv5 network, and determine an image for the cropped olecranon region as a region of interest (ROI). For example, referring to FIG. 4, (a) of FIG. 4 corresponds to an original X-ray image and (b) corresponds to an image segmented for the olecranon region. The region segmentation unit 120 may detect a bone portion corresponding to the olecranon by applying YOLOv5, the first step of the cascade model, to an X-ray image such as (a) and acquire an image by segmenting only the region corresponding to the olecranon as in (b). That is, the region segmentation unit 120 crops only the olecranon region portion from the X-ray image through the YOLOv5 network and extracts the cropped olecranon region as a region of interest. Based on the region of interest extracted here, Step S230 to be described below may be performed.

In one embodiment, the region segmentation unit 120 may detect a region corresponding to the olecranon by applying YOLOv5 to an X-ray image, which is an input image, and segment the region corresponding to the olecranon by using EfficientDet. Here, EfficientDet is a deep learning algorithm modified based on the backbone of EfficientNet model, and a segmentation layer is added so that EfficientDet may be used when the region segmentation unit 120 segments the olecranon region.

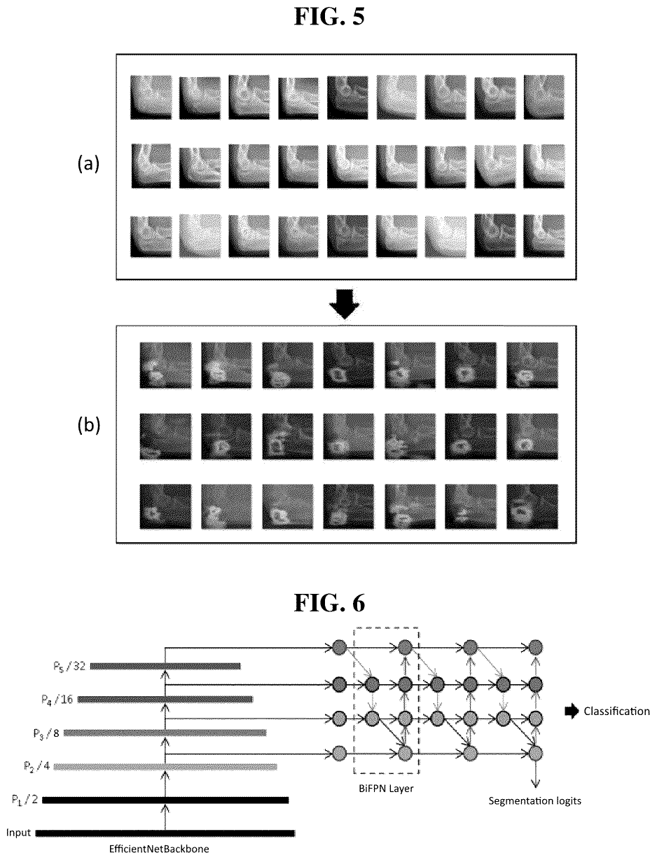

The morphological analysis unit 130 analyzes the morphological features of the olecranon ossification center in the segmented olecranon region (Step S230). Preferably, the morphological analysis unit 130 may determine the region for the olecranon ossification center by applying a deep learning algorithm to the olecranon region and determine the morphological features of the olecranon ossification center. Referring to FIG. 3, the morphological analysis unit 130 may classify the morphological features of the olecranon ossification center by using EfficientDet, which is a cascade-trained deep learning algorithm. That is, the morphological analysis unit 130 applies EfficientDet to the segmented olecranon region to classify the olecranon region and the apophysis. Here, EfficientDet is a network that achieves high efficiency and accuracy by using complex scaling that simultaneously adjusts depth, width, and resolution, and is advantageous in precisely identifying objects for elbow shapes that are different for each person. For example, referring to FIG. 5, (a) of FIG. 5 corresponds to the olecranon region acquired through the region segmentation unit 120, and (b) of FIG. 5 corresponds to the region for the olecranon ossification center, which is a reference for determining the morphological features of the olecranon ossification center. The morphological analysis unit 130 applies EfficientDet, which is a deep learning algorithm, to the olecranon region such as (a) to determine the region for the olecranon ossification center of the olecranon region such as (b) and determine the morphological features of the region for the olecranon ossification center. That is, EfficientDet, which is a deep learning algorithm, analyzes the morphological features based on the highlighted region illustrated in (b) of FIG. 5.

More specifically, EfficientDet, a deep learning algorithm applied to the region segmentation unit 120, the morphological analysis unit 130, and the bone age determination unit 140 to be described below, is configured to be suitable for the present disclosure by bringing in the backbone of the EfficientNet model, as illustrated in FIG. 6, the Efficient-Net located on the left is used as the backbone, the EfficientNet of the backbone is applied as b4 (that is, EfficientNet-b4), and four Bi-directional Feature Pyramid Network (BiFPN) layers are applied as a feature network at the last step, and the box network is removed from the class network that classifies the class of the object and the box network that generates the bounding box that determines the location and size of the object, and a segmentation layer is added. Here, b4 of EfficientNet-b4 is a number applied according to the depth of the layer, such as b1, b2, or b4, and the depth of the layer may be applied or changed variously. This deep learning algorithm has an additional segmentation prediction logit that predicts X-ray images on a pixel basis, and the segmentation prediction logit may predict the bone region in the X-ray image on a pixel basis. Using this modified EfficientDet has the advantage of lowering the error rate and enabling faster calculation speeds because each box does not have to be checked.

Referring to FIG. 7, the Efficient-Net, which is a structure included in the backbone of EfficientDet and used as a backbone network, consists of multiple Convolution (Conv) layers, MBconv blocks, conv 1×1, and pooling & FC (Fully Connected) layers. In Conv 3×3, a 3×3 kernel using 32 channels and one Conv layer are stacked, and then one MBConv1 block using a 3×3 kernel and 16 channels, two MBConv6 blocks using a 3×3 kernel and 24 channels, two MBConv6 blocks using a 5×5 kernel and 40 channels, and three MBConv6 blocks using a 3×3 kernel and 80 channels are connected. Here, MBConv6 performs depth-wise batch normalization and swish process again in MBconv. Finally, a Conv layer using a 1×1 kernel, a pooling layer, and a fully connected layer which is a dense layer are stacked. Since the data input to the deep learning algorithm is a 2D X-ray image, not a 3D z-axis deep image, layers are stacked in units of 4 in this way: Input->P1/2->P2/4->P3/8->P4/16->P5/32, and the accuracy of object detection may be improved by integrating various scales of features through the BiFPN layer. This corresponds to a network structure in which segmentation logits are attached to the layer passing through the BiFPN layer, segmentation of the elbow area, and classification of the area in the final step are performed. At this time, the layers may be stacked more deeply as necessary. Here, segmentation corresponds to the segmentation that identifies the exact boundary of the olecranon area and distinguishes the region from other regions through the region segmentation unit 120. The region segmentation unit 120 may also segment the olecranon area using EfficientDet, and the classification corresponds to the bone age determination performed through the bone age determination unit 140 described below.

The bone age determination unit 140 determines the bone age based on the morphological features of the ossification center of the olecranon analyzed in Step S230 (Step S240). Preferably, the bone age determination unit 140 may classify the olecranon bone age according to the preset age-specific olecranon characteristic criteria based on the morphological features of the olecranon ossification center by applying EfficientDet, which is a deep learning algorithm. Here, the deep learning algorithm is the same as the algorithm applied in Step S230.

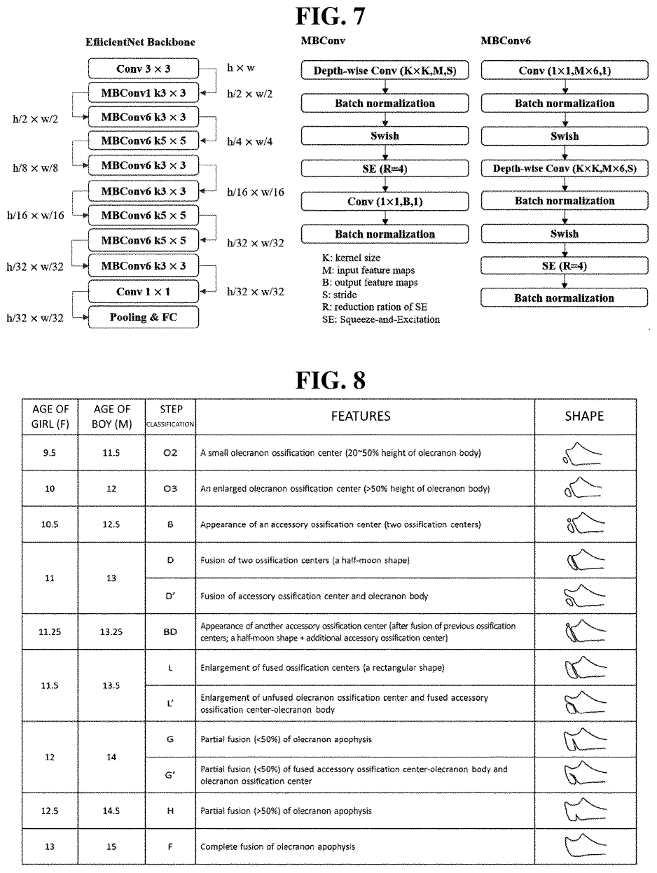

Preferably, the preset age-specific olecranon characteristic criteria may be set in 6-month units from 9.5 to 13 years old in girls, and in 6-month units from 11.5 to 15 years old in boys. Here, the olecranon characteristic criteria may be further set for 11.25 years old, which is the interval between 11 and 11.5 years old in girls, and 13.25 years old, which is the interval between 13 and 13.5 years old in boys. Referring to FIG. 8, it may be seen that the olecranon characteristics and shape by age of the girl (F) and the boy (M) are set as preset age-specific olecranon characteristic criteria. In 11-year-old girls and 13-year-old boys, 11.5-year-old girls and 13.5-year-old boys, and 12-year-old girls and 14-year-old boys, two forms corresponding to D step and D′ step, L step and L′ step, and G step and G′ step may appear, respectively, which correspond to the forms that may appear according to the order of fusion during the ossification process.

More specifically, the preset age-specific olecranon characteristic criteria may be classified into the first ossification process and the second ossification process according to the order of fusion of the ossification center of the olecranon and accessory ossification center. That is, when the ossification center of the olecranon and accessory ossification center fuse first after the appearance of the accessory ossification center in the ossification process, it may proceed as the first ossification process, and when the accessory ossification center and the olecranon body fuse first, it may proceed as the second ossification process.

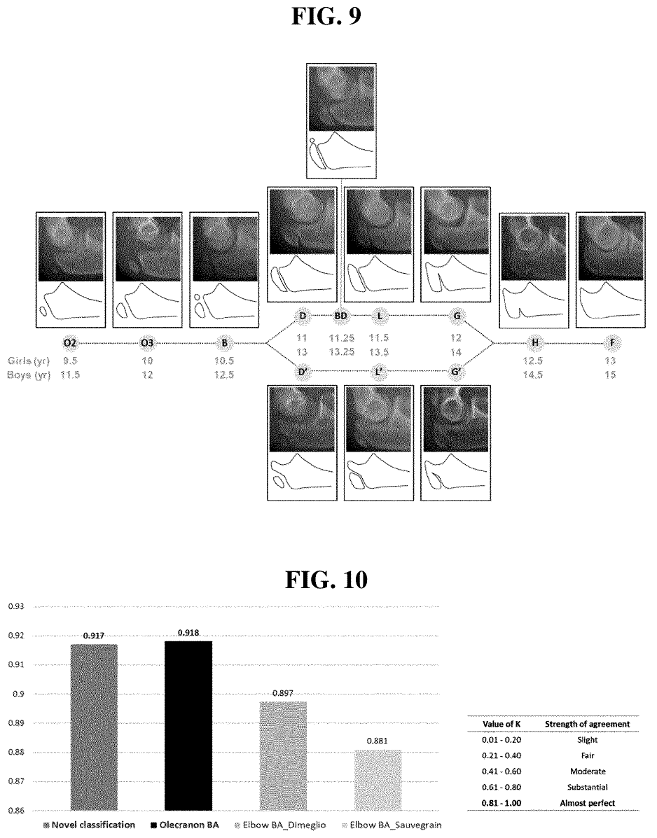

First, referring to FIG. 9, the first ossification process may include
Step O2 [A small ossification center (20 to 50% height of olecranon body)] corresponding to 9.5 years old in girls and 11.5 years old in boys,
Step O3 [An enlarged olecranon ossification center (>50% height of olecranon body)] corresponding to 10 years old in girls and 12 years old in boys,
Step B [Appearance of an accessory ossification center (two ossification centers)] corresponding to 10.5 years old in girls and 12.5 years old in boys,
Step D [Fusion of two ossification centers (a half-moon shape)] corresponding to 11 years old in girls and 13 years old in boys,
Step L [Enlargement of fused ossification centers (a rectangular shape)] corresponding to 11.5 years old in girls and 13.5 years old in boys,
Step G [Partial fusion (<50%) of olecranon apophysis] corresponding to 12 years old in girls and 14 years old in boys,
Step H [Partial fusion (>50%) of the olecranon apophysis] corresponding to 12.5 years old in girls and 14.5 years old in boys, and
Step F [Complete fusion of olecranon apophysis] corresponding to 13 years old in girls and 15 years old in boys.

Next, referring to FIG. 9, the second ossification process may include
Step O2 [A small ossification center (20 to 50% height of olecranon body)] corresponding to 9.5 years old in girls and 11.5 years old in boys,
Step O3 [enlarged olecranon ossification center (>50% height of olecranon body)] corresponding to 10 years old in girls and 12 years old in boys,
Step B [Appearance of an accessory ossification center (two ossification centers)] corresponding to 10.5 years old in girls and 12.5 years old in boys,
Step D′ (Fusion of accessory ossification center and olecranon body) corresponding to 11 years old in girls and 13 years old in boys,
Step L′ [Enlargement of unfused olecranon ossification center and fused accessory ossification center-olecranon body] corresponding to 11.5 years old in girls and 13.5 years old in boys,
Step G′ [Partial fusion (<50%) of fused accessory ossification center-olecranon body and olecranon ossification center] corresponding to 12 years old in girls and 14 years old in boys,
Step H [Partial fusion (>50%) of the olecranon apophysis] corresponding to 12.5 years old in girls and 14.5 years old in boys, and
Step F [Complete fusion of olecranon apophysis] corresponding to 13 years old in girls and 15 years old in boys.

Here, Step BD [Appearance of another accessory ossification center (after fusion of previous ossification centers; a half-moon shape+additional accessory ossification center)] corresponding to 11.25 years old in girls and 13.25 years old in boys may be further included between Step D and Step L of the first ossification process and Step D′ and Step L′ of the second ossification process.

That is, the bone age determination unit 140 determines the bone age as the age corresponding to the step where the morphological features of the center of the olecranon ossification acquired through the morphological analysis unit 130 match each step of the preset age-specific olecranon characteristic criteria.

FIG. 10 is a diagram for explaining the performance of the bone age assessment method according to one embodiment.

Referring to FIG. 10, it illustrates an inter-observer agreement of two pediatric radiologists, and it may be seen that the case where the bone age assessment method according to the present disclosure (Novel classification and Olecranon Bone Age (Olecranon BA)) is applied is higher than Elbow BA_Dimeglio and Elbow BA_Sauvegrain which are the existing olecranon bone age assessment methods. Here, the Novel classification illustrates the result analyzed based on the step expressed as 02 to F, and Olecranon BA illustrates the result analyzed based on age. Since Step D and Step D′, Step L and Step L′, and Step G and Step G′ have the same bone age but are classified differently, they were analyzed separately.

In addition, referring to [Table 1] below, it may be seen that the bone age assessment method according to the present disclosure shows high reliability when compared with Sauvegrain (Elbow BA_Sauvegrain) and Dimeglio (Elbow BA_Dimeglio) which are the existing elbow bone age assessment methods, and Greulich-Pyle and Tanner-Whitehouse hybrid (Hand BA_GP/TW3) and Korean pediatric standard bone age (Hand BA_KS) which are the existing hand and wrist bone age assessment methods.

TABLE 1

| | | | Girls | | Boys | |
|---|---|---|---|---|---|---|
| | | | ICC | 95% CI | ICC | 95% CI |
| Olecranon BA | vs. | Elbow BA_Sauvegrain | 0.965 | 0.962-0.968 | 0.966 | 0.957-0.974 |
| Olecranon BA | vs. | Elbow BA_Dimeglio | 0.978 | 0.976-0.980 | 0.978 | 0.971-0.983 |
| Olecranon BA | vs. | Hand BA_GP/TW3 | 0.825 | 0.811-0.839 | 0.887 | 0.860-0.910 |
| Olecranon BA | vs. | Hand BA_KS | 0.891 | 0.883-0.899 | 0.915 | 0.891-0.931 |
| Elbow BA_Sauvegrain | vs. | Elbow BA_Dimeglio | 0.927 | 0.919-0.935 | 0.943 | 0.923-0.958 |

In addition, the AI model, which is a deep learning algorithm applied to the bone age assessment method according to the present disclosure, illustrates high accuracy and low error rate. Specifically, in the case of the internal dataset of the novel classification, it illustrates accuracy of 0.96, sensitivity of 0.80, and specificity of 0.98, and in the case of the external dataset thereof, it illustrates accuracy of 0.87, sensitivity of 0.76, and specificity of 0.91. In addition, in the case of the olecranon bone age, in the case of the internal validation, it illustrates MSE of 0.088 and RMSE of 0.296, and in the case of the external validation, it illustrates MSE of 0.125 and RMSE of 0.398. Here, the internal validation is used to assess how well the algorithm model or the classification method applied to perform the bone age assessment method fits the learning data, and the external validation is used to assess how well the algorithm model, or the classification method generalizes to new data.

Meanwhile, the steps of the method or algorithm described in connection with the embodiments of the present disclosure may be implemented directly in hardware, implemented as a software module executed by hardware, or implemented by a combination of these. The software module may reside in a Random Access Memory (RAM), a Read Only Memory (ROM), an Erasable Programmable ROM (EPROM), an Electrically Erasable Programmable ROM (EEPROM), a flash memory, a hard disk, a removable disk, a CD-ROM, or any form of computer-readable recording medium well known in the art to which the present disclosure pertains.

The components of the present disclosure may be implemented as a program (or application) to be executed by combining with a computer as hardware and stored on a medium. The components of the present disclosure may be executed as software programming or software elements, and similarly, the embodiments may be implemented in a programming or scripting language such as C, C++, Java, assembler, or the like, including various algorithms implemented as a combination of data structures, processes, routines, or other programming elements. Functional aspects may be implemented as algorithms that are executed on one or more processors.

Although the preferred embodiments of the bone age assessment method and device according to the present disclosure have been described above, the present disclosure is not limited thereto, and various modifications may be made and implemented within the scope of the claims, the detailed description of the disclosure, and the attached drawings, which also belong to the present disclosure.

### REFERENCE SIGNS LIST

100: Bone age assessment device
110: Image acquisition unit
120: Region segmentation unit
130: Morphological analysis unit
140: Bone age determination unit
150: Control unit

1. A bone age assessment method performed by a bone age assessment device, the bone age assessment method comprising:
(a) acquiring a lateral elbow X-ray image;
(b) segmenting an olecranon region from the X-ray image;
(c) analyzing morphological features of an olecranon ossification center in the segmented olecranon region; and
(d) determining a bone age based on the analyzed morphological features.

2. The bone age assessment method of claim 1, wherein the (b) includes
predicting a bone region in the X-ray image on a pixel basis, and
segmenting a region corresponding to the olecranon in the X-ray image according to a result of the pixel-based prediction.

3. The bone age assessment method of claim 2, wherein the (c) includes
determining a region for the olecranon ossification center in the olecranon region, and
determining the morphological features of the olecranon ossification center.

4. The bone age assessment method of claim 3, wherein (d) includes classifying the olecranon bone age according to a preset age-specific olecranon characteristic criteria based on the morphological features of the olecranon ossification center.

5. The bone age assessment method of claim 4, wherein the preset age-specific olecranon characteristic criteria are set in 6-month units from 9.5 to 13 years old in girls, and in 6-month units from 11.5 to 15 years old in boys.

6. The bone age assessment method of claim 5, wherein in the preset age-specific olecranon characteristic criteria, the olecranon characteristic criteria are further set for 11.25 years olds in girls and 13.25 years old in boys.

7. The bone age assessment method of claim 4, wherein the preset age-specific olecranon characteristic criteria are classified into a first ossification process and a second ossification process according to the order of fusion of an accessory ossification center and the ossification center of the olecranon.

8. The bone age assessment method of claim 7, wherein the first ossification process includes a small olecranon ossification center step in which a height of an olecranon body is 20% or more and less than 50%, an enlarged olecranon ossification center step in which the height of the olecranon body is 50% or more, an appearance step of an accessory ossification center, a fusion step of the olecranon ossification center and the accessory ossification center, an enlargement step of the fused ossification center, a partial fusion step of less than 50% of an olecranon apophysis, a partial fusion step of 50% or more of the olecranon apophysis, and a complete fusion step of the olecranon apophysis.

9. The bone age assessment method of claim 8, further comprising an appearance step of another accessory ossification center after the fusion of the olecranon ossification center and the accessory ossification center between the fusion step of the olecranon ossification center and the accessory ossification center and the enlargement step of the fused ossification center.

10. The bone age assessment method of claim 7, wherein the second ossification process includes a small olecranon ossification center step in which the height of the olecranon body is 20% or more and less than 50%, an enlarged olecranon ossification center step in which the height of the olecranon body is 50% or more, an appearance step of an accessory ossification center, a fusion step of the accessory ossification center and the olecranon body, an enlargement step of the fused accessory ossification center-olecranon body and the unfused olecranon ossification center, a partial fusion step of less than 50% of the olecranon ossification center and the fused accessory ossification center-olecranon body, a partial fusion step of 50% or more of the olecranon apophysis, and a complete fusion step of the olecranon apophysis.

11. The bone age assessment method of claim 10, further comprising an appearance step of another accessory ossification center after the fusion of the olecranon ossification center and the accessory ossification center between the fusion step of the accessory ossification center and the olecranon body and the enlargement step of the fused accessory ossification center-olecranon body and the unfused olecranon ossification center.

12. A bone age assessment device comprising:
an image acquisition unit for acquiring a lateral elbow X-ray image;
a region segmentation unit for segmenting an olecranon region from the X-ray image;
a morphological analysis unit for analyzing morphological features of an olecranon ossification center in the segmented olecranon region; and
a bone age determination unit for determining bone age based on the analyzed morphological features.

13. The bone age assessment device of claim 12, wherein the region segmentation unit predicts a bone region in the X-ray image on a pixel basis and segments the region corresponding to the olecranon in the X-ray image according to a result of the pixel-based prediction.

14. The bone age assessment device of claim 13, wherein the morphological analysis unit determines a region for the olecranon ossification center in the olecranon region, and determines the morphological features of the olecranon ossification center.

15. The bone age assessment device of claim 14, wherein the bone age determination unit classifies the olecranon bone age according to a preset age-specific olecranon characteristic criteria based on the morphological features of the olecranon ossification center.

16. The bone age assessment device of claim 15, wherein the preset age-specific olecranon characteristic criteria are set in 6-month units from 9.5 to 13 years old in girls, and in 6-month units from 11.5 to 15 years old in boys.

17. The bone age assessment device of claim 16, wherein in the preset age-specific olecranon characteristic criteria, the olecranon characteristic criteria are further set for 11.25 years olds in girls and 13.25 years old in boys.

18. The bone age assessment device of claim 15, wherein
the preset age-specific olecranon characteristic criteria are classified into a first ossification process and a second ossification process according to the order of fusion of the accessory ossification center and the ossification center of the olecranon.

19. A computer program stored in a computer-readable medium, wherein when a command of the computer program is executed, a method according to claim 1 is performed.
